SEQUENCE LISTING (1 sequence)

SEQ ID NO: 1 - DNA, 1000 bp, synthetic construct
  source 1..1000: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

(19) United States
(12) Patent Application Publication
(10) Pub. No.: US 2024/0150791 A1
(43) Pub. Date: May 9, 2024

(54) PROMOTER FOR SPECIFIC EXPRESSION OF GENES IN ROD PHOTORECEPTORS

(71) Applicant: Institute of Molecular and Clinical Ophthalmology Basel (IOB), Basel (CH)

(72) Inventors: Botond ROSKA, Oberwil (CH); Hendrik P.N. SCHOLL, Binningen (CH); Josephine JUETTNER, Allschwil (CH)

(21) Appl. No.: 18/543,981

(22) Filed: Dec. 18, 2023

Related U.S. Application Data
(63) Continuation of application No. PCT/EP21/68653, filed on Jul. 6, 2021.

(51) Int. Cl. C12N 15/86; A61K 48/00

(52) U.S. Cl. CPC C12N 15/86; A61K 48/0058; C12N 2750/14143; C12N 2830/008; C12N 2830/48

(57) ABSTRACT

An isolated nucleic acid molecule, comprising a first nucleic acid sequence having promoter activity for the specific expression of an exogenous gene in rod photoreceptor cells and at least one further nucleic acid sequence encoding the exogenous gene, operably linked to said promoter, wherein said first nucleic acid sequence comprises or consists of SEQ ID NO:1 or of a functionally equivalent sequence variant thereof, having at least 80% sequence identity to SEQ ID NO:1.

### RELATED APPLICATIONS

The present application is a continuation of International Application No. PCT/EP2021/068653, filed on Jul. 6, 2021, the content of which are hereby incorporated by reference in their entirety.

### REFERENCE TO A SEQUENCE LISTING XML

This application contains a Sequence Listing which has been submitted electronically in XML format. The Sequence Listing XML is incorporated herein by reference. Said XML file, created on Dec. 11, 2023, is named 761131_00005_SL and is 4,096 bytes in size.

### FIELD OF THE INVENTION

The present invention relates to the field of treatment and prevention of ocular diseases associated with photoreceptor cell degeneration. The present invention provides a nucleic acid sequence having promoter activity for the expression of therapeutic genes of interest specifically in rod photoreceptor cells of the human or animal retina.

### BACKGROUND OF THE INVENTION

Blindness disables millions of people worldwide and is a major health problem. One most common cause of blindness is the dysfunction of the retina and in particular of the photoreceptor cells. Most common forms of retinal blindness are Retinitis Pigmentosa (RP) and macular degeneration (AMD) inter alia causing a degeneration of photoreceptors cells and the consequent loss of light sensitivity. There is a need to be able to obviate the problems associated with such degeneration of photoreceptors or loss in their light sensitivity. This can preferably be effected by the expression of therapeutic molecules, in particular, polypeptides, kinines, trophic factors, channels, opsins or receptors, or of nucleic acid molecules, specifically in the rod photoreceptor cells.

For expression, recombinant genes or exogenous or heterologous genes are usually transfected into the target cells, cell populations or tissues, typically as cDNA constructs in the context of an active expression cassette to allow transcription of the heterologous gene. The DNA construct is recognized by the cellular transcription machinery in a process that involves the activity of many trans-acting transcription factors (TF) at cis-regulatory elements, including enhancers, silencers, insulators and promoters, herein all generally referred to as “regulatory elements”. Gene promoters are involved in all of these levels of regulation, serving as the determinant in gene transcription by integrating the influences of the DNA sequence, transcription factor binding and epigenetic features. They determine the strength of, for example, transgene expression as well as in which cell type or types said transgene will be expressed.

Common promoters used for driving heterologous gene expression in mammalian cells are the human and mouse cytomegalovirus (CMV) major immediate early promoter. They confer a strong expression and have proved robust in several cell types. Other viral promoters such as the SV40 immediate early promoter and the Rous Sarcoma Virus (RSV) long-terminal-repeat (LTR) promoter are also used frequently in expression cassettes. Instead of viral promoters, cellular promoters can also be used. Among known promoters are those from house-keeping genes that encode abundantly transcribed cellular transcripts, such as beta-actin, elongation factor 1-alpha (EF-1alpha), or ubiquitin. Compared to viral promoters, eukaryotic gene expression is more complex and requires a precise coordination of many different factors.

Aspects of concern regarding the use of endogenous regulatory elements for transgene expression include the generation of stable mRNA, and that expression can take place in the native environment of the host cell where trans-acting transcription factors are provided accordingly. Since expression of eukaryotic genes is controlled by a complex machinery of cis-and trans-acting regulatory elements, most cellular promoters suffer from a lack of extensive functional characterization. Parts of the eukaryotic promoter are usually located immediately upstream of its transcribed sequence and serves as the point of transcriptional initiation. The core promoter immediately surrounds the transcription start site (TSS) which is sufficient to be recognized by the transcription machinery. The proximal promoter comprises the region upstream of the core promoter and contains the TSS and other sequence features required for transcriptional regulation. Transcription factors act sequence-specific by binding to regulatory motifs in the promoter and enhancer sequence.

Some promoters can act in a cell specific manner and can be used to express a transgene in cells of a specific type or in cells of a particular subset.

One objective of the present invention is to obtain new promoter sequences and genetic tools comprising these promoter sequences, that are suitable for use in recombinant constructs to express one or more desired genes in mammalian (human or animal) cells at high expression levels. In particular, it is an objective to provide new promoters that can express desired genes at high levels in a rod photoreceptor cell specific manner and for use in the therapy and/or prophylaxis of ocular disorders, including disorders characterized by degeneration of rod photoreceptors. Rod specific promoters are also desired for use as a research tool, for example to develop systems for the study of neurodegenerative disorders, vision restoration, drug discovery, and diagnosis of disorders connected to the degeneration of rod photoreceptors.

### SUMMARY OF THE INVENTION

In a first aspect, the present invention provides a nucleic acid molecule which has promoter activity for the specific expression of an exogenous gene in rod photoreceptor cells. The nucleic acid sequence of one specific nucleic acid molecule is:

(SEQ ID NO: 1)
AACCCAAGAAATTACAGGCTGAAACCAGAAAAGAACACATTAAAGCAC
CAAGAGAAAGTTGGAGTGGGTTGAAGGGAAACAGATTTTTAAAGTTAA
GGCTCTGTGAAATGGGTAGAATTAACTACAGGTTAAAAATAAAATGTT
AACTAAAGGTTGCCTCTGAGTAACAGGATTATGGGTGATTTTAATTGT
CTTCTTTGTGTATGTTCAACAGTGACTATAATATGTATTACTTTTGGA
ATAAAGGAAAACCTGAAAGGTGTGTTGTTTTATAAGGGCCCTTAGGTT
GCCAAAATTAGAGTCATTGAAATCTAAAGCTGATAAAAACTTTAGTGC
AAAGATTGTGACATGGGAGACTACACATACCAGATCCATAATGTACAT
GAGGACAGTAGGCCGAGGGGCCCTGCACATTGAAAGCCCACATGGGAG
AAGCCCTTGGGAAGGGGAGTGGAAGGATGAGGCAAGGGGCCGGGGGGA
TGCAGAGGCTGGCAGGCAGTCATTTCTCAGCTTCAGCCATTCCCGCCA
TGGGGGAATGTGGACAGAGAAGCCAAACAAATCTCCTAAACAGTAAAT
GTCAGTCTTCTGTGTCAGATATTTAAGAAAACTAACAGAGGTCAGAGA
AGACACACCTACAGCAAGTAGACTGTCCCTGTGCTGCCTTTTTGCAAC
CCCTGCTTTGGCAGTGCTCAAGCCCACCTCCTGCTCTGTGCAGACATC
TCTTCTTTGCTCTTACTAGACCAAGGTGAAAGAAAACTCTCACCTTCT
CCCATCTGGCCCCACAGCATCTGGAACACACTGATCCTCATAATCCTT
GTTCTTGAGAAATATTAATGACTTAATCTCCCAAGCTTGCTCCCTCTC
CTGTGCAGGCCATCTCAGTATGTTTTGCAGACAAGACCCAGAGAAGTC
CAGACTGGACTTGTTGCAGACTGCAAAACTGCCATTGGAAGGCCTCCG
TCCCAGTCCTTCTACAGAGTAGCCAGTGGGATTCCCAGCC.

The promoter sequence SEQ ID NO:1, particularly if embedded into a viral vector or other expression system, drives specific and efficient gene expression in rod photoreceptors of mice, organoids and humans. This allows basic and preclinical research specific manipulation of rod photoreceptors in economic model systems as in vivo mouse retina and in vitro human retinal organoids to discover retinal disease mechanisms and develop new gene therapy approaches. The ability to use of the same viral vector on human retina allows direct translation of these results and will therefore accelerate the application of novel gene therapies in the clinic.

The present invention hence provides nucleic acid molecules conferring promoter activity for the specific expression of an exogenous gene in rod photoreceptor cells wherein the nucleic acid molecules have a nucleic acid sequence comprising or consisting of SEQ ID NO:1 or of a functionally equivalent sequence variant thereof, having at least 80% sequence identity to SEQ ID NO:1, the functionally equivalent sequence variant having promoter activity for the specific expression of an exogenous gene in rod photoreceptor cells. Preferably, the nucleic acid conferring promoter activity is operably linked to an exogenous gene that is heterologous.

In one embodiment, the functional variant of the invention has more than 80% sequence identity to SEQ ID NO:1. In a preferred embodiment, the functional variant has at least 90% sequence identity to SEQ ID NO:1. In another preferred embodiment, the functional variant has at least 95% sequence identity to SEQ ID NO:1. In another preferred embodiments, the functional variant has at least 96%, or at least 97%, or at least 98% sequence identity to SEQ ID NO:1.

The sequence variants can be derived from the sequence of SEQ ID NO:1 by way of deletion or insertion or replacement of one or more base in the sequence, Preferably, the sequence variant of the invention is derived from the sequence of SEQ ID NO:1 by way of deletion, insertion and/or replacement of 1 to 200 bp, or of 1 to 100 bp, or of 1 to 50 bp, or of 1 to 20 bp.

In some embodiments, the nucleic acid sequence having promoter activity for the specific expression of an exogenous gene in rod photoreceptor cells shares over a sequence length (consecutive nucleotides) of at least 400 bp, or at least 500 bp, or at least 600 bp, or at least 700 bp, or at least 800 bp, or at least 900 bp sequence identity to the sequence of SEQ ID NO:1.

In some embodiments, the nucleic acid sequence having promoter activity for the specific expression of an exogenous gene in rod photoreceptor cells is at least 800 bp, at least 900 bp, at least 950 bp, at least 880 bp. In preferred variants thereof the sequence is not more than 1200 bp, or not more than 1100 bp, or not more than 1050 bp, or not more than 1020 bp. In some embodiments, said nucleic acid sequence has a sequence length of 800 to 1200 bp, preferably 800 to 1000 bp.

In an embodiment, the promoter of the invention comprises, or consists of, a functional variant of SEQ ID NO: 1 selected from the group consisting of
a sequence having at least 80% identity to SEQ ID NO: 1,
a sequence comprising at least 100 consecutive nucleotides of SEQ ID NO: 1, and
a sequence which is capable of hybridizing under low, medium or high stringency conditions with the nucleic acid sequence of SEQ ID NO: 1 or its complementary strand.

In a particular embodiment, the promoter of the invention comprises, or consists of, a functional variant having at least 80, 81, 82, 83, 84, 85, 86, 87, 88, 89, 90, 91, 92, 93, 94, 95, 96, 97, 98 or 99% identity to SEQ ID NO: 1, preferably over the entire sequence of SEQ ID NO: 1. The promoter of the invention may differ from the polynucleotide of SEQ ID NO: 1 by 1, 2, 3, 4, 5, 6, 7, 8, 9, 10, 11, 12, 13, 14 or 15 substitutions, deletions and/or insertions.

In a particular embodiment, the promoter of the invention comprises, or consists of, a functional variant having a sequence comprising at least 100, 200, 300, 400, 500, 600, 700, 800, 900 consecutive nucleotides of SEQ ID NO: 1. Preferably, it comprises, or consists of, a functional variant having a sequence comprising at least 500 consecutive nucleotides of SEQ ID NO: 1.

In a further particular embodiment, the promoter of the invention comprises, or consists of, a functional variant having a sequence capable of hybridizing under low, medium or high stringency conditions with the nucleic acid sequence of SEQ ID NO: 1 or its complementary strand, preferably under medium stringency conditions, more preferably under high stringency conditions.

Also provided is an isolated nucleic acid molecule comprising a sequence that hybridizes under medium stringent conditions to an isolated nucleic acid molecule of the invention as described above or its complementary strand. Also provided is an isolated nucleic acid molecule comprising a sequence that hybridizes under highly stringent conditions to an isolated nucleic acid molecule of the invention as described above or its complementary strand.

One particular embodiment of the invention is an isolated nucleic acid molecule, comprising or consisting of: a first nucleic acid sequence conferring promoter activity for the specific expression of an exogenous gene in rod photoreceptor cells wherein said first nucleic acid sequence comprises or consists of SEQ ID NO:1 or of a functional variant thereof, and at least one further nucleic acid sequence encoding the exogenous gene, operably linked to said rod photoreceptor specific promoter. Preferably, the nucleic acid conferring promoter activity is operably linked to an exogenous gene that is heterologous.

Thus, in a second aspect of the invention, there is provided a nucleic acid molecule that particularly resembles a gene expression cassette. The expression cassette of the invention may comprise one or more nucleic acids operably linked to the promoter of the invention. The at least one further nucleic acid sequence encodes for one or more of: therapeutically effective proteins, peptides or nucleic acids, or a reporter molecule. For example, the promoter may be operably linked to one or more therapeutic genes and a nucleic acid encoding a reporter protein or to a therapeutic gene and an optogenetic actuator. Typically, the expression cassette includes at least one nucleic acid that is heterologous to the promoter. In particular, the therapeutic protein is selected from the group consisting of: MT-ND4, MT-ND1, MT-ND6, MT-CYB, MT-C03, MT-ND5, MT-ND2, MT-COI, MT-ATP6, MT-ND4L, OPA1, OPA3, OPA7 and AC02.

In particular, the therapeutic protein is a neurotrophic factor, preferably selected from the group consisting of GDNF, VEGF, CNTF, FGF2, BDNF and EPO.

In particular, the therapeutic protein is an anti-apoptotic protein, preferably selected from the group consisting of BCL2 and BCL2L1.

In particular, the therapeutic protein is an anti-angiogenic factor, preferably selected from the group consisting of endostatin, angiostatin and sFIt.

In particular, the therapeutic protein is an anti-inflammatory factor preferably selected from the group consisting of IL10, IL1R1, TGFBI and IL4.

In particular, the therapeutic protein is the rod-derived cone viability factor (RdCVF).

In particular, the therapeutic protein is an optogenetic activator, preferably selected from the group consisting of rhodopsins, photopsins, melanopsins, pinopsins, parapinopsins, VA opsins, peropsins, neuropsins, encephalopsins, retinochromes, RGR opsins, microbial opsins with red-shifted spectral properties such as ReaChR, Chrimson or ChrimsonR, vertebrate opsins that can recruit Gi/0 signalling such as short wavelength vertebrate opsin or long wavelength vertebrate opsin, channelrhodopsins from microalgae of the genus Chlamydomonas such as channelrhodopsin-1 and channelrhodopsin-2 (from Chlamydomonas reinhardtii), and variants thereof.

In particular, the therapeutic protein is an optogenetic inhibitor, preferably selected from the group consisting of halorhodopsins such as halorhodopsin (NpHR), enhanced halorhodopsins (eNpHR2.0 and eNpHR3.0) and the red-shifted halorhodopsin Halo57, archaerhodopsin-3 (AR-3), archaerhodopsin (Arch), bacteriorhodopsins such as enhanced bacteriorhodopsin (eBR), proteorhodopsins, xanthorhodopsins, Leptosphaeria maculans fungal opsins (Mac), the cruxhalorhodopsin Jaws, and variants thereof.

In particular, the therapeutic nucleic acid is preferably selected from the group consisting of a siRNA, a shRNA a RNAi, a miRNA, an antisense RNA, a ribozyme and a DNAzyme.

In particular, the reporter protein is preferably selected from the group consisting of fluorescent proteins, calcium indicators, alkaline phosphatases, beta-galactosidases, beta-lactamases, horseradish peroxidase, and variants thereof. Preferred reporter tag is selected from mCitrine, EYFP, and tdTomato.

In a further aspect, the present invention relates to a viral vector for the specific expression of an exogenous gene in rod photoreceptor cells. The vector comprises the expression cassette of the present invention. More preferably, the vector is a retroviral vector, in particular a lentiviral vector or a non-pathogenic parvovirus. In another embodiment, the vector is an adeno-associated viral (AAV) vector or an AAV-derived hybrid vector and may comprise two ITRs flanking the nucleic acid encoding the polypeptide or nucleic acid of interest.

In a further aspect, the present invention relates to a viral particle, in particular comprising an AVV-derived capsid and the AAV vector of the invention. In preferred embodiments, the AAV-derived c apsid is selected from the group consisting of AAV-2, AAV-5, AAV-7m8 (AAV2-7m8), AAV-BP21, AAV-PHP.B, AAV-PHP.eB, AAV-44.9, AAV-NHP26 (LB26), AAV9-7m8, AAV5, AAV8, and AAV9 serotype capsid.

In a further aspect, the invention relates to a cell, or host cell, preferably a rod photoreceptor cell, which is transformed with an expression cassette of the invention, with a vector or a viral particle of the invention.

In a further aspect, the invention relates to a pharmaceutical composition comprising an expression cassette of the invention, and a pharmaceutically acceptable carrier or excipient.

In one embodiment thereof, the pharmaceutical composition comprises a vector of the invention, and a pharmaceutically acceptable carrier or excipient.

In a further embodiment thereof, the pharmaceutical composition comprises a viral particle of the invention, and a pharmaceutically acceptable carrier or excipient.

In a further embodiment thereof, the pharmaceutical composition comprises a transformed cell of the invention and a pharmaceutically acceptable carrier or excipient.

In another aspect, the present invention relates to an expression cassette, a vector, a viral particle, or a host cell of the invention, for use in the treatment or prevention of an ocular disease in a human or mammalian animal. Preferably, the ocular disease is selected from diseases associated with photoreceptor cell degeneration or loss or loss in light sensitivity, such as age-related macular degeneration, rod-cone dystrophy, rod dysfunction, Leber congenital amaurosis, Stargardt's disease, diabetic retinopathy, Best's disease, Retinitis Pigmentosa, Choroideremia or a tapetoretinal degeneration.

In a further aspect, the present invention relates to the use of a nucleic acid, expression cassette, vector or viral particle of the invention for the expression of a therapeutically effective protein, peptide or nucleic acid, or a reporter molecule in rod photoreceptor cells.

In a further aspect, the present invention relates to a method of expressing a therapeutically effective protein, peptide or nucleic acid, or a reporter molecule in rod photoreceptor cells, preferably for the specific transformation of a rod photoreceptor cell with a therapeutically effective protein, peptide or nucleic acid, or a reporter molecule, comprising the step of incubating or putting into contact the rod photoreceptor cell with an expression cassette of the invention, a vector of the invention, a viral particle of the invention, a transformed cell of the invention, or a pharmaceutical composition of the invention.

Alternatively or in addition, the method of the invention comprises the step of introducing into the rod photoreceptor cell an expression cassette of the invention, a vector of the invention, a viral particle of the invention, a transformed cell of the invention, or a pharmaceutical composition of the invention.

In a further aspect, the present invention relates to a therapeutic method for the prevention or treatment of an ocular disease related to photoreceptor degeneration or loss or loss in light sensitivity, comprising the step of administering to a human individual or animal in need thereof a therapeutically active dose the pharmaceutical composition of the invention. In a preferred embodiment, the pharmaceutical composition of the invention is administered to the vitreous of the eye to be treated. In another preferred embodiment, the pharmaceutical composition of the invention is administered to the anterior chamber of the eye to be treated. In another preferred embodiment the pharmaceutical composition of the invention is administered to the anterior chamber of the eye to be treated. In another preferred embodiment, the pharmaceutical composition of the invention is administered to the subretinal space of the retina of the eye to be treated.

In a further aspect, the present invention relates to a kit for expressing a gene in rod photoreceptors comprising an isolated nucleic acid molecule according of the invention.

### BRIEF DESCRIPTION OF THE DRAWINGS

FIG. 1A: Sequence map of the constructed plasmid for AAV generation: pAAV-SEQ ID NO:1-CatCh-GFP

FIG. 1B: Sequence map of the constructed plasmid for AAV generation for expression in human retina organoids: pAAV-SEQ ID NO:1-GFP.

FIG. 2: Spinning disk confocal microscope images of cross-sections of human retina, retinal organoid and whole mount mouse retina infected with AAV-SEQ ID NO:1-Catch-GFP and AAV-SEQ ID NO:1-GFP, respectively. Left: CatCh-GFP/GFP (green); middle-left, immunostaining with cone marker CAR (magenta); middle-right: CatCh-GFP/GFP and cone marker; right: CatCh-GFP/GFP and cone marker and nuclear stain (Hoechst, white).

FIG. 3: Confocal image of AAV-infected human and mouse retina (top views) and whole human retinal organoid cross-section, CatCh-GFP/GFP (black).

FIG. 4: Quantification plots for expression in human retina (top row), human retinal organoids (middle row) and mouse retina (lower row): left col.: quantification of CatCh-GFP/GFP+ cell density as a percentage of rod photoreceptor density; values are the mean ±s.e.m. from n=12 confocal images; right col:: quantification of AAV-targeting specificity shown as a percentage of the major (black) and minor (gray) cell types or classes among cells expressing CatCh-GFP/GFP.

### DETAILED DESCRIPTION OF THE INVENTION

Rod photoreceptors, rod cells, or rods, are photoreceptor cells in the retina of the eye that can function in less intense light than the other type of visual photoreceptor, cone cells. Rods are concentrated at the outer edges of the retina and are used in peripheral vision. On average, there are approximately 90 million rod cells in the human retina. More sensitive than cone cells, rod cells are almost entirely responsible for night vision. However, because they have only one type of light-sensitive pigment, rather than the three types that human cone cells have, rods have little, if any, role in color vision.

The nucleic acid molecules of the invention conferring promoter activity which allows a very for specific expression of an exogenous gene in rod photoreceptor cells, but not in other cells, which are no rod photoreceptor cells. In particular, the nucleic acid molecules of the invention have significant promoter activity at high cell specificity for human rod photoreceptor cells in organoid cell cultures reconstituted from isolated human cells, but have no promotor activity in all other cells of this organoid cell culture. Moreover, the nucleic acid molecules of the invention have significant promoter activity at high cell specificity for human rod photoreceptor cells in isolated human retina explants, but have no promotor activity in all other cells of this retina explant. The nucleic acid molecules of the invention advantageously allow for the provision of specific screening tools to screen for transgenes or other pharmaceutically compounds to compensate for a genetic defect in retinal cells, specifically rod photoreceptor cells, and eventually to provide and establish one or more therapeutically effective compounds to protect from, ameliorate or cure the retinal degeneration or blindness related to such a genetic defect.

The methods using promoters of the invention can also be used for in vitro testing of vision restoration, said method comprising the steps of contacting rod photoreceptors expressing one or more transgene under the control of a promoter of the invention with an agent, and comparing at least one output obtained after the contact with said agent with the same output obtained before said contact with said agent. The methods using nucleic acid sequence of the invention can be used for identifying therapeutic agents for the treatment of a neurological disorder or of a disorder of the retina involving rod photoreceptors, said method comprising the steps of contacting a test compound with rod photoreceptors expressing one or more transgene under a promoter of the invention, and comparing at least one output of rod photoreceptors obtained in the presence of said test compound with the same output obtained in the absence of said test compound.

Moreover, the nucleic acid molecules of the invention confer promoter activity for a very specific expression of an exogenous gene, in particular coding for a therapeutically active effector, in rod photoreceptor cells of the human retina in a patient. At the same time, the nucleic acid molecules of the invention prevent expression of said exogenous gene or effector in retinal cells other than rod photoreceptor cells. The nucleic acid molecules of the invention advantageously allow for the provision of a specific expression of therapeutic effector in the human eye to protect from, ameliorate or cure retinal degeneration or blindness related to a genetic defect or disorder in the rod photoreceptor cell or to a genetic defect or disorder in a cell functionally connected to the rod photoreceptor cells, in particular the retinal pigment epithelium (RPE) cell.

“Specific expression” of an exogenous gene, also referred to as “expression only in a certain type of cell” means that at least more than 75% of the cells expressing the gene of interest are of the type specified, i.e. rod photoreceptors in the present case.

The synthetic promoter of the invention can drive expression of an exogenous gene of interest in human rod photoreceptors in a human retina at high specificity, i.e. at an expression specificity of more than 80%, preferably more than 85.0% or more than 86.0%. At the same time, the synthetic promoter of the invention can drive expression of the exogenous gene of interest in murine rod photoreceptors in a mouse retina at high specificity, i.e. at an expression specificity of more than 90%, preferably more than 95%. Moreover, the synthetic promoter of the invention can drive expression of an exogenous gene of interest in rod photoreceptors in human retinal organoids at high specificity, i.e. at an expression specificity of more than 90%, preferably more than 98.0%. Here and in the following, expression specificity is quantified by determining the percentage of rod photoreceptors expression the gene of interest in the overall cell population expression the gene of interest.

More specifically, the synthetic promoter of the invention can drive expression of an exogenous gene of interest in human rod photoreceptors at high rate, i.e. at an expression density of more than 25.0%, preferably more than 30.0%. Moreover, the synthetic promoter of the invention can drive expression of an exogenous gene of interest in murine rod photoreceptors at an expression density of more than 35.0%, preferably more than 40.0%. Here and in the following, expression density is defined as the percentage density of rod photoreceptors expressing the exogenous gene of interest relative to published mean density of rod photoreceptors (cells/mm2) in humans (Jonas, J. B. et al. Count and density of human retinal photoreceptors. Graefes Arch. Clin. Exp. Ophthalmol. 230, 505-510 (1992)) and in the murine retina, respectively. The synthetic promoter SEQ ID NO: 1 drove expression in 30.4% of total human rod photoreceptors and 41.2% of total murine rod photoreceptors.

Moreover, the synthetic promoter of the invention can drive expression of an exogenous gene of interest in human rod photoreceptors in retinal organoids obtained from isolated human cells, at an expression density of more than 30%, preferably more than 34%.

One of the main advantages of the promoter sequence of the invention is its small size. Indeed, the promoter of the invention has a length of less 1.2 kb and is thus particularly suitable for use in AAV vectors wherein the DNA payload is severely limited.

In some embodiments, the promoter of the invention is not operably linked to SNCG gene, and in particular to the human SNCG gene. In some other embodiments, the promoter of the invention is not operably linked to a gene encoding a reporter protein, and in particular to a gene encoding luciferase. Preferably, the promoter of the invention is not operably linked to SNCG gene or to a gene encoding luciferase. In a second aspect, the present invention relates to an expression cassette comprising a promoter of the invention operably linked to a nucleic acid of interest.

The nucleic acid operably linked to the promoter of the invention may encode a polypeptide of interest or a nucleic acid of interest.

Preferably, the promoter of the invention is operably linked to a heterologous nucleic acid. As used herein, the term “heterologous” means a nucleic acid other than the nucleic acid that the promoter is operably linked to in a naturally occurring genome. In an embodiment, the nucleic acid operably linked to the promoter of the invention encodes a polypeptide of interest. The polypeptide of interest may be any polypeptide of which expression in rod photoreceptor cells is desired. In particular, the polypeptide of interest may be a therapeutic polypeptide, reporter protein or optogenetic actuator. In an embodiment, the nucleic acid operably linked to the promoter of the invention is a therapeutic gene, i.e. encodes a therapeutic polypeptide.

Examples of therapeutic genes include, but are not limited to, nucleic acids for replacement of a missing or mutated gene known to cause retinal disease such as MT-ND4 (Gene ID: 4538), MT-ND1 (Gene ID: 4535), MT-ND6 (Gene ID: 4541), MT-CYB (Gene ID: 4519), MT-C03 (Gene ID: 4514), MT-ND5 (Gene ID: 4540), MT-ND2 (Gene ID: 4536), MT-COI (Gene ID: 4512), MT-ATP6 (Gene ID: 4508), MT-ND4L (Gene ID: 4539), OPA1 (Gene ID: 4976), OPA3 (Gene ID: 80207), OPA7 (Gene ID: 84233), ACO2 and (Gene ID: 50). The therapeutic gene may also encode neurotrophic factors such as GDNF (Gene ID: 2668), CNTF (Gene ID: 1270), FGF2 (Gene ID: 2247), BDNF (Gene ID: 627) and EPO (Gene ID: 2056), anti-apoptotic genes such as BCL2 (Gene ID: 596) and BCL2L1 (Gene ID: 598), anti-angiogenic factors such as endostatin, angiostatin and sFIt, anti-inflammatory factors such as IL10 (Gene ID: 3586), IL1R1 (Gene ID: 3554), TGFBI (Gene ID; 7045) and IL4 (Gene ID: 3565), or the rod-derived cone viability factor (RdCVF) (Gene ID: 115861).

Additional signal peptide may be added to therapeutic proteins, in particular in order to import them inside certain organelles (such as mitochondria), to secrete them from the cell, or to insert them into cellular membrane.

In another embodiment, the polypeptide of interest is an optogenetic actuator, which is a photochemically reactive polypeptide that uses vitamin A or isoforms thereof as its chromophore. An optogenetic actuator in particular is a light-gated ion pump or channel that absorbs light and is activated by light. The optogenetic actuator may be from a prokaryotic organism or a eukaryotic organism. In particular, it may be a microbial opsin or a vertebrate opsin. The optogenetic actuator may be an optogenetic activator or an optogenetic inhibitor.

An optogenetic activator causes a cell to depolarize upon exposure to light. Examples of optogenetic activators include rhodopsins, photopsins, melanopsins, pinopsins, parapinopsins, VA opsins, peropsins, neuropsins, encephalopsins, retinochromes, RGR opsins, microbial opsins with red-shifted spectral properties such as ReaChR, Chrimson or ChrimsonR, vertebrate opsins that can recruit Gi/0-signalling such as short wavelength vertebrate opsin or long wavelength vertebrate opsin, channelrhodopsins from microalgae of the genus Chlamydomonas such as channelrhodopsin-1, channelrhodopsin-2 (from Chlamydomonas reinhardtii), and optimized or functionally improved variants (e.g. codon optimized variants, mutants, chimeras) thereof.

In a more particularly preferred embodiment, the optogenetic actuator is an optogenetic activator, preferably selected from channelrhodopsins, ChrimsonR and variants thereof, and more preferably selected from hChR2 (L132C)-hCatCh and ChrimsonR-tdTomato.

In a more particularly preferred embodiment, the optogenetic actuator is an optogenetic activator, preferably selected from channelrhodopsins and variants thereof, and more preferably is hChR2 (L132C)-hCatCh.

An optogenetic inhibitor causes a cell to hyperpolarize upon exposure to light. Examples of optogenetic inhibitors include, but are not limited to, halorhodopsins such as halorhodopsin (NpHR), enhanced halorhodopsins (eNpHR2.0 and eNpHR3.0) and the red-shifted halorhodopsin Halo57, archaerhodopsin-3 (AR-3), archaerhodopsin (Arch), bacteriorhodopsins such as enhanced bacteriorhodopsin (eBR), proteorhodopsins, xanthorhodopsins, Leptosphaeria maculans fungal opsins (Mac), the cruxhalorhodopsin Jaws, and optimized or functionally improved variants (e.g. codon optimized variants, mutants, chimeras) thereof.

In a further embodiment, the nucleic acid operably linked to the promoter of the invention encodes a reporter protein. Preferably, the reporter protein is detectable in living rod photoreceptor cells. The expression of a reporter protein under the control of a promoter of the invention allows specifically detecting or identifying rod photoreceptor cells. The reporter protein may be, for example, a fluorescent protein (e.g., GFP), calcium indicator (e.g. GCamP), luciferase, alkaline phosphatase, beta-galactosidase, beta-lactamase, horseradish peroxidase, and variants thereof. In a particular embodiment, the reporter protein is selected from the group consisting of fluorescent proteins, calcium indicators, alkaline phosphatases, beta-galactosidases, beta-lactamases, horseradish peroxidase, and variants thereof.

In another embodiment, the nucleic acid operably linked to the promoter of the invention encodes a nucleic acid of interest. The nucleic acid of interest may be any nucleic acid of which expression in rod photoreceptor cells is desired. In particular, the nucleic acid of interest may be a therapeutic nucleic acid. The nucleic acid may be, for example, an siRNA, an shRNA an RNAi, a miRNA, an antisense RNA, a ribozyme or a DNAzyme. In a particular embodiment, the nucleic acid encodes an RNA that when transcribed from the nucleic acid operably linked to the promoter of the invention can treat or prevent an ocular disease by interfering with translation or transcription of an abnormal or excess protein associated with said disorder. For example, the nucleic acid of interest may encode for an RNA, which treats the disease by highly specific elimination or reduction of mRNA encoding the abnormal and/or excess proteins.

Preferably, the vector of the invention is a vector suitable for use in gene or cell therapy, and in particular is suitable to target rod photoreceptor cells.

The vector of the invention is preferably a viral genome vector including any element required to establish the expression of the polypeptide of interest in a host cell such as, for example, a promoter, e.g., a promoter of the invention, an ITR, a ribosome binding element, terminator, enhancer, selection marker, intron, polyA signal, and/or origin of replication.

In some embodiments, the vector is a viral vector, such as vectors derived from Moloney murine leukemia virus vectors (MoMLV), MSCV, SFFV, MPSV or SNV, lentiviral vectors (e.g. derived from human immunodeficiency virus (HIV), simian immunodeficiency virus (SIV), feline immunodeficiency virus (FIV), bovine immunodeficiency virus (BIV) or equine infectious anemia virus (EIAV)), adenoviral (Ad) vectors, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors.

In particular embodiments, the vector is a retroviral vector, preferably a lentiviral vector or a non-pathogenic parvovirus. As known in the art, depending on the specific viral vector considered for use, suitable sequences should be introduced in the vector of the invention for obtaining a functional viral vector, such as AAV ITRs for an AAV vector, or LTRs for lentiviral vectors.

As used herein, the term “AAV vector” refers to a polynucleotide vector comprising one or more heterologous sequences (i.e., nucleic acid sequence not of AAV origin) that are flanked by at least one AAV inverted terminal repeat sequence (ITR), preferably two ITRs. Such AAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins).

An “inverted terminal repeat” or “ITR” sequence is a term well understood in the art and refers to relatively short sequences found at the termini of viral genomes which are in opposite orientation. An “AAV inverted terminal repeat (ITR)” sequence is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contain several shorter regions of self-complementarity (designated A, A′, B, B′, C, C and D regions), allowing intra-strand base-pairing to occur within this portion of the ITR. AAV ITRs for use in the vectors of the invention may have a wild-type nucleotide sequence or may be altered by the insertion, deletion or substitution. The serotype of the inverted terminal repeats (ITRs) of the AAV vector may be selected from any known human or nonhuman AAV serotype.

When an AAV vector is incorporated into a larger polynucleotide (e.g., in a chromosome or in another vector such as a plasmid used for cloning or transfection), then the AAV vector may be referred to as a “pro-vector” which can be “rescued” by replication and encapsidation (i.e. the enclosure of viral nucleic acid within a capsid) in the presence of AAV packaging functions and suitable helper functions. The AAV vector of the invention can be in any of a number of forms, including, but not limited to, plasmids, linear artificial chromosomes, complexed with lipids, encapsulated within liposomes, and encapsidated in a viral particle, e.g., an AAV particle. The promoter or expression cassette of the invention may be introduced into the vector by any method known by the skilled person.

The vector may further comprise one or more nucleic acid sequences encoding selectable marker such as auxotrophic markers (e.g., LEU2, URA3, TRP 1 or HIS3), detectable labels such as fluorescent or luminescent proteins (e.g., GFP, eGFP, DsRed, CFP), or protein conferring resistance to a chemical/toxic compound (e.g., MGMT gene conferring resistance to temozolomide). These markers can be used to select or detect host cells comprising the vector and can be easily chosen by the skilled person according to the host cell.

All the embodiments of promoter and expression cassette of the invention are also contemplated in this aspect. The vector of the invention may be packaged into a virus capsid to generate a “viral particle”. Thus, in a further aspect, the present invention also relates to a viral particle comprising a vector of the invention. In a particular embodiment, the vector is an AAV vector and is packaged into an AAV-derived capsid to generate an “adeno-associated viral particle” or “AAV particle”. Thus, used herein, the term “AAV particle” refers to a viral particle composed of at least one AAV capsid protein and an encapsidated AAV vector genome.

The capsid serotype determines the tropism range of the AAV particle. Multiple serotypes of adeno-associated virus (AAV), including 12 human serotypes and more than 100 serotypes from nonhuman primates have now been identified (Howarth al., 2010, Cell Biol Toxicol 26: 1-10). Among these serotypes, human serotype 2 was the first AAV developed as a gene transfer vector. Other currently used AAV serotypes include, but are not limited to, AAV1, AAV3, AAV4, AAV5, AAV6, AAV7, AAV8, AAVrh8, AAV9, AAV10, AAVrhIO, AAV11, AAV 12, AAVrh74 and AAVdj, etc. In addition, non-natural engineered variants and chimeric AAV can also be useful. In particular, the capsid proteins may be variants comprising one or more amino acid substitutions enhancing transduction efficiency.

Different AAV serotypes are used to optimize transduction of particular target cells or to target specific cell types within a particular target tissue (e.g., rod photoreceptor cells). An AAV particle can comprise viral proteins and viral nucleic acids of the same serotype or any natural or artificial sequence variant of AAV. For example, the AAV particle may comprise AAV2 capsid proteins and at least one, preferably two, AAV2 ITR. Any combination of AAV serotypes for production of an AAV particle is provided herein as if each combination had been expressly stated herein. In preferred embodiment, the AAV particle comprises an AAV-derived capsid selected from the group consisting of AAV2, AAV-5, AAV-7m8 (AAV2-7m8, Dalkara et al. Sci Transl Med (2013), 5, 189ra76), AAV9 or AAV8 capsid. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus.

Alternatively, to using AAV natural serotypes, artificial AAV serotypes may be used in the context of the present invention, including, without limitation, AAV with a non-naturally occurring capsid protein. Such an artificial capsid may be generated by any suitable technique, using a selected AAV sequence (e.g., a fragment of a VP1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid or a mutated AAV capsid. A chimeric capsid comprises VP capsid proteins derived from at least two different AAV serotypes or comprises at least one chimeric VP protein combining VP protein regions or domains derived from at least two AAV serotypes.

Capsid proteins may also be mutated, in particular to enhance transduction efficiency. Mutated AAV capsids may be obtained from capsid modifications inserted by error prone PCR and/or peptide insertion or by including one or several amino acids substitutions. In particular, mutations may be made in any one or more of tyrosine residues of natural or non-natural capsid proteins (e.g. VP1, VP2, or VP3). Preferably, mutated residues are surface exposed tyrosine residues. Exemplary mutations include but are not limited to tyrosine-to-phenylalanine substitutions such as Y252F, Y272F, Y444F, Y500F, Y700F, Y704F, Y730F, Y275F, Y281F, Y508F, Y576F, Y612G, Y673F and Y720F. In a preferred embodiment, the AAV particle comprise an AAV2-derived capsid. In this embodiment, the capsid may comprise one or more tyrosine-to-phenylalanine substitutions, preferably comprises Y444F substitution.

In addition, the genome vector (i.e. a vector of the invention) of the AAV particle may either be a single stranded or self-complementary double-stranded genome. Self-complementary double-stranded AAV vectors are generated by deleting the terminal resolution site (trs) from one of the AAV terminal repeats. These modified vectors, whose replicating genome is half the length of the wild type AAV genome have the tendency to package DNA dimers. In a preferred embodiment, the AAV particle implemented in the practice of the present invention has a single stranded genome.

AAV production cultures for the production of AAV virus particles all require; 1) suitable host cells, including, for example, human-derived cell lines such as HeLa, A549, or HEK293 cells, 2) suitable helper virus function, provided by wild-type or mutant adenovirus, e.g. temperature sensitive adenovirus, Herpes virus, or a plasmid construct providing helper functions; 3) AAV rep and cap genes and gene products; 4) a nucleic acid of interest flanked by at least one AAV ITR sequences, e.g., a vector of the invention; and 5) suitable media and media components to support AAV production that are well-known in the art. Host cells for producing AAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from HEK293, A549 or HeLa cells.

In another aspect, the present invention also relates to an isolated host cell transformed or transfected with an expression cassette, vector or viral particle of the invention. The host cell may be any animal cell, plant cell, bacterium cell or yeast. Preferably, the host cell is a mammalian cell or an insect cell. More preferably, the host cell is a human cell. In preferred embodiments, the host cell is a retinal ganglion cell, in particular a human rod photoreceptor cell. The expression cassette or vector of the invention may be transferred into host cells using any known technique including viral infection, and may be maintained in the host cell in an ectopic form or may be integrated into the genome. In preferred embodiments, the expression cassette or vector of the invention is transferred into the host cell by viral infection, preferably using a viral particle of the invention, more preferably using an AAV particle of the invention.

The major routes of delivery of the vector to the retina in an animal model or human are intravitreal injection and subretinal surgical delivery. Intravitreal injection is an established and safe route for vector administration to the eye. Here, the vector is injected directly into the vitreous. With subretinal injection, the vector is delivered in a localized subretinal bleb between the retinal pigment epithelium (RPE) and the photoreceptor layer in a surgical procedure. This is accomplished in a practical approach during pars plana vitrectomy. Subretinal administration provides the most direct access to photoreceptors and the RPE. A third approach is the delivery of the vector into the anterior section, in particular the anterior chamber of the eye.

When working with isolated retina, optimal viral delivery for retinal cells can be achieved by mounting the ganglion cell side downwards, so that the photoreceptor side of the retina is exposed and can thus be better transfected. Another technique is slicing, for example, with a razor blade, the inner limiting membrane of the retina, such that the delivering viruses can penetrate the inner membranes. A further way is to embed the retina in agar, slicing said retina and applying the delivery viruses from the side of the slice.

Any source of retinal cells can be used for the present invention. In some embodiments of the invention, the retinal cells come from, or are in, a human retina. In other embodiments, the retina is from an animal, e.g. of rodent origin. Human retina can be easily obtained from cornea banks where said retinas are normally discarded after the dissection of the cornea. Adult human retina has a large surface (about 1100 mm²) and can therefore be easily separated to several experimentally subregions.

The present invention also relates to a pharmaceutical composition comprising an expression cassette, vector, viral particle or cell of the invention. Such compositions comprise a therapeutically effective amount of the therapeutic agent (an expression cassette, vector, viral particle or cell of the invention), and a pharmaceutically acceptable excipient. As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered.

As is well known in the art, pharmaceutically acceptable excipients are relatively inert substances that facilitate administration of a pharmacologically effective substance and can be supplied as liquid solutions or suspensions, as emulsions, or as solid forms suitable for dissolution or suspension in liquid prior to use. For example, an excipient can give form or consistency, or act as a diluent. Suitable excipients include but are not limited to stabilizing agents, wetting and emulsifying agents, salts for varying osmolality, encapsulating agents, pH buffering substances, and buffers. Such excipients include any pharmaceutical agent suitable for direct delivery to the eye which may be administered without undue toxicity. Pharmaceutically acceptable excipients include, but are not limited to, sorbitol, any of the various tween compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Most preferably, the composition is combined with saline, Ringer's balanced salt solution (pH 7.4), and the like.

The composition is formulated to be administered to the eye, in particular by intraocular injection, e.g., by subretinal and/or intravitreal administration. For intravitreal delivery, the pharmaceutical composition of the invention is injected directly into the vitreous. For subretinal delivery, the pharmaceutical composition of the invention is delivered in a localized subretinal bleb between the retinal pigment epithelium (RPE) and the photoreceptor layer in a surgical procedure. This is accomplished in a practical approach during pars plana vitrectomy (ppV). Subretinal administration provides the most direct access to photoreceptors and the RPE. A third approach is the delivery of the pharmaceutical composition into the anterior section of the eye, in particular into the anterior chamber.

The amount of pharmaceutical composition to be administered may be determined by standard procedure well known by those of ordinary skill in the art. Physiological data of the patient (e.g. age, size, and weight) and type and severity of the disease being treated have to be taken into account to determine the appropriate dosage.

The compounds of the pharmaceutical composition may be formulated for administration by injection, e. g., by subretinal or intravitreal injection. Formulations for injection may be presented in unit dosage form, e. g., in ampoules or in multi-dose containers. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e. g., sterile pyrogen-free water, before use.

In addition to the formulations described previously, the compounds of the pharmaceutical composition may also be formulated as a depot preparation or for use in an implanted delivery system. Such long-acting formulations may be administered by implantation, for example, intraocular, or by intraocular injection. The compounds may also be formulated as a depot preparation for use in an implanted drug delivery system or device, particularly for repeated refill of a reservoir in the implanted drug delivery system or device. Thus, the compounds may be formulated with suitable polymeric or hydrophobic materials (for example, as an emulsion in an acceptable oil) or ion exchange resins, or as sparingly soluble derivatives, for example, as a sparingly soluble salt.

The pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

In an embodiment, the pharmaceutical composition comprises a vector or viral particle of the invention, more preferably an AAV vector or particle. In another embodiment, the pharmaceutical composition comprises host cells of the invention, preferably human host cell of the invention, i.e. transformed or transfected with an expression cassette, vector or viral particle of the invention, preferably with an AAV particle. Optionally, the composition comprising host cells may be frozen for storage at any temperature appropriate for storage of the cells. In a particular embodiment, the composition comprises viral particles of the invention and each unit dosage comprises from 10E+8 to 10E+13 viral particles.

The pharmaceutical composition may further comprise one or several additional active compounds such as corticosteroids, antibiotics, analgesics, immunosuppressants, trophic factors, or any combinations thereof.

The invention also provides kits for carrying out the therapeutic regimens of the invention. Such kits comprise in one or more containers therapeutically or prophylactically effective amounts of the compositions in pharmaceutically acceptable form.

The composition in a vial of a kit may be in the form of a pharmaceutically acceptable solution, e. g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e. g., saline, dextrose solution, etc.), preferably sterile, to reconstitute the complex to form a solution for injection purposes.

In another embodiment, a kit further comprises a needle or syringe, preferably packaged in sterile form, for injecting the complex, and/or a packaged alcohol pad. Instructions are optionally included for administration of compositions by a clinician or by the patient.

The method of the invention may also further comprise administering at least one additional therapeutic agent to the subject. In particular, said therapeutic agent may be selected from the group consisting of a corticosteroid, an antibiotic, an analgesic, an immunosuppressant, or a trophic factor, or any combinations thereof.

The composition of the invention may be administered before or after the disease becomes symptomatic, e.g., before or after partial or complete rod photoreceptor cell or photoreceptor cell degeneration and/or before or after partial or complete loss of vision.

Thus, in another aspect, the present invention also relates to a non-human animal model comprising an expression cassette, vector, viral particle or host cell of the invention.

Such animal model may be used for in vivo studies of rod photoreceptor cell functions. Using the promoter, cassette, vector or viral particle of the invention, it is possible to identify or track rod photoreceptor cell, or monitor their activity, for example through expression of reporter proteins or voltage or calcium sensitive proteins. The non-human animal model may also be used in screening methods for identifying or selecting pharmaceutical agent acting on rod photoreceptor cells. Preferably, the non-human animal model is a mammal, more preferably a primate, rodent, rabbit or mini pig.

The promoter, expression cassette or vector may be maintained in cells of this model in an episomic form or may be integrated into its genome. Methods for transfecting or transforming animal cells or for producing transgenic animals expressing a nucleic acid sequence of interest under to control of a chosen promoter, i.e. the promoter of the invention, is well known by the skilled person and may be easily adapted according to cells and animals.

### Definitions

As used herein, the term “promoter” refers to any cis-regulatory elements that are generally located upstream (towards the 5′ region) of the gene and that define where transcription of the gene by RNA polymerase begins. RNA polymerase and the necessary transcription factors bind to the promoter and initiate transcription of the gene. In the context of the present invention, the promoter leads to the specific expression of genes operably linked to them in rod photoreceptors.

As used herein, the term “promoter activity” refers to the ability of a promoter to initiate transcription of a nucleic acid to which it is operably linked. Promoter activity can be measured using procedures known in the art or as described in the Examples. For example, promoter activity can be measured as an amount of mRNA transcribed by using, for example, Northern blotting or polymerase chain reaction (PCR). Alternatively, promoter activity can be measured as an amount of translated protein product, for example, by Western blotting, ELISA, colorimetric assays and various activity assays, including reporter gene assays and other procedures known in the art or as described in the Examples.

The term “operably linked” refers, as used herein, to the association of nucleic acid sequences on a single nucleic acid molecule so that the function of one is affected by the other. For example, a promoter is operably linked with a coding sequence when it is capable of affecting the expression of that coding sequence, i.e., the coding sequence is under the transcriptional control of the promoter.

As used herein, the term “nucleic acid” or “polynucleotide” refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. “Polynucleotides” can be composed of single-and double-stranded DNA, DNA that is a mixture of single-and double-stranded regions, single-and double-stranded RNA, and RNA that is mixture of single-and double-stranded regions, hybrid molecules comprising DNA and RNA that may be single-stranded or, more typically, double-stranded or a mixture of single-and double-stranded regions. In addition, polynucleotides can be composed of triple-stranded regions comprising RNA or DNA or both RNA and DNA. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH2) or a mixed phosphoramidate-phosphodiester oligomer. The nucleic acid of the invention can be prepared by any method known to one skilled in the art, including chemical synthesis, recombination, and mutagenesis. In preferred embodiments, the nucleic acid of the invention is a DNA molecule, preferably synthesized by recombinant methods well known to those skilled in the art. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, the term “isolated nucleic acid” refers to a nucleic acid molecule which has been identified and separated and/or recovered from a component of its natural environment. In particular, this term refers to a nucleic acid molecule which is separated from other nucleic acid molecules which are present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid molecule is free of sequences which naturally flank the nucleic acid molecule in the genomic DNA of the organism from which the nucleic acid molecule is derived.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g. , a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition.

As used herein, the term “variant” refers to a nucleotide sequence differing from the original sequence, but retaining essential properties thereof. Generally, variants are overall closely similar, and, in many regions, identical to the original polynucleotide. The sequence of the variant may differ by nucleotide substitutions, deletions or insertions of one or more nucleotides in the sequence, which do not impair the promoter activity. The variant may have the same length of the original sequence, or may be shorter or longer.

The term “functional variant” refers to a variant of SEQ ID NO: 1 that exhibits a promoter activity of SEQ ID NO: 1, i.e. that exhibits a promoter activity specific of rod photoreceptor cells.

As used herein, the term “sequence identity” or “identity” refers to the number (%) of matches (identical nucleic acid residues) in positions from an alignment of two polynucleotide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent nucleic acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/). Those skilled in the art can determine appropriate parameters for measuring alignment, including any algorithms needed to achieve maximal alignment over the full length of the sequences being compared. For purposes herein, % nucleic acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty—1, Joining Penalty—30, Randomization Group Length=0, Cutoff Score=I, Gap Penalty—5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

As used herein, the term “low stringency conditions” means for probes of at least 100 nucleotides in length, prehybridization and hybridization at 42° C. in 5×SSPE, 0.3% SDS, 200 micrograms/mL sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/mL sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

The term “high stringency conditions” means for probes of at least 100 nucleotides in length, prehybridization and hybridization at 42° C. in 5×SSPE, 0.3% SDS, 200 micrograms/mL sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

The expression “encoding for” as in “polynucleotide encoding for a polypeptide” encompasses a polynucleotide which includes only coding sequence for the polypeptide as well as a polynucleotide which includes additional coding and/or non-coding sequence.

As used herein, the term “expression cassette” refers to a nucleic acid construct comprising a coding sequence and one or more control sequences required for expression of said coding sequence. In particular, one of these control sequences is a promoter of the invention. Generally, the expression cassette comprises a coding sequence and regulatory sequences preceding (5′ non-coding sequences) and following (3′ non-coding sequences) the coding sequence that are required for expression of the selected gene product. Thus, an expression cassette typically comprises a promoter sequence, a coding sequence and a 3′ untranslated region that usually contains a polyadenylation site and/or transcription terminator. The expression cassette may also comprise additional regulatory elements such as, for example, enhancer sequences, a polylinker sequence facilitating the insertion of a DNA fragment within a vector and/or splicing signal sequences. The expression cassette is usually included within a vector, to facilitate cloning and transformation.

The term “gene” means the segment of DNA involved in producing a polypeptide chain; it includes regions preceding and following the coding region “leader and trailer” as well as intervening sequences (introns) between individual coding segments (exons).

As used herein, the term “therapeutic gene” refers to a gene encoding a therapeutic protein which is useful in the treatment of a pathological condition. The therapeutic gene, when expressed, confers a beneficial effect on the cell or tissue in which it is present, or on a patient in which the gene is expressed. Examples of beneficial effects include amelioration of a sign or symptom of a condition or disease, prevention or inhibition of a condition or disease, or conferral of a desired characteristic. Therapeutic genes include genes that partially or wholly correct a genetic deficiency in the patient. In particular, the therapeutic gene may be, without limitation, a nucleic acid sequence encoding a protein useful in gene therapy to relieve deficiencies caused by missing, defective or sub-optimal levels of said protein in a cell or tissue of a subject. The therapeutic polypeptide may, e.g., supply a polypeptide and/or enzymatic activity that is absent, defective or present at a sub-optimal level in rod photoreceptor cells, supply a polypeptide and/or enzymatic activity that indirectly counteracts an imbalance in rod photoreceptor cells. The therapeutic polypeptide may also be used to reduce the activity of a polypeptide by acting, e.g., as a dominant-negative polypeptide. Preferably, the therapeutic polypeptide supplies a polypeptide and/or enzymatic activity that is absent, defective or present at a sub-optimal level in rod photoreceptor cells, more preferably a polypeptide and/or enzymatic activity that is absent or defective in rod photoreceptor cells.

A “virus” is a sub-microscopic infectious agent that is unable to grow or reproduce outside a host cell. Each viral particle, or virion, consists of genetic material, DNA or RNA, within a protective protein coat called a capsid. The capsid shape varies from simple helical and icosahedral (polyhedral or near-spherical) forms, to more complex structures with tails or an envelope. Viruses infect cellular life forms and are grouped into animal, plant and bacterial types, according to the type of host infected.

As used herein, the term “vector” refers to a nucleic acid molecule used as a vehicle to transfer genetic material, and in particular to deliver a nucleic acid into a host cell, either in vitro or in vivo. Vectors include, but are not limited to, plasmids, phasmids, cosmids, transposable elements, viruses, and artificial chromosomes (e.g., YACs).

As used herein, the term “treatment”, “treat” or “treating” refers to any act intended to ameliorate the health status of patients such as therapy, prevention, prophylaxis, and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease. In particular, the term “treatment of an ocular disease” may refer to a treatment to provide enhanced vision, to prevent progression of the disease to total blindness, to prevent spread of damage to uninjured ocular cells, to improve damage in injured ocular cells, to prevent the occurrence of retinal damage or to rescue eyes having mild or advanced disease. In some embodiments, this term refers to a treatment to prevent, reduce or stop rod photoreceptor cells degeneration by providing a therapeutic protein correcting a genetic deficiency of the patient. In some other embodiments, this term refers to a treatment to reanimate the retina or restore vision, using optogenetics.

By a “therapeutically efficient amount” is intended an amount of pharmaceutical composition of the invention administered to a subject that is sufficient to constitute a treatment as defined above of ocular disease.

### EXAMPLES

### Vector Construct and Production

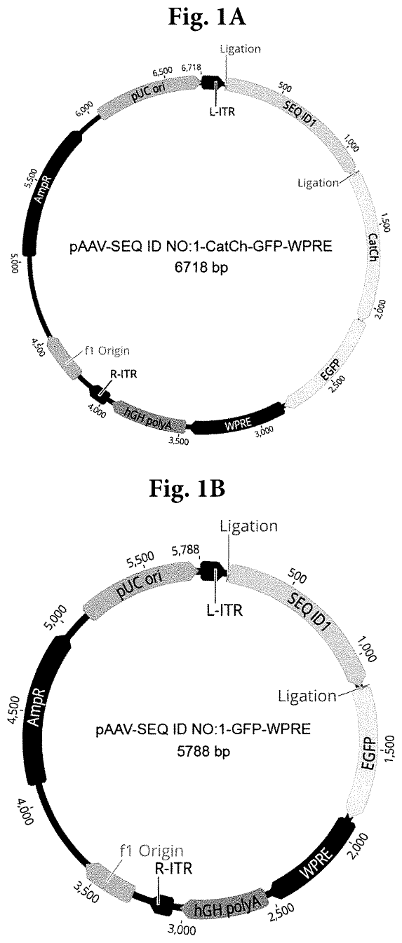

A nucleic acid molecule with the sequence of SEQ ID NO:1 was chemically synthesized by GenScript Inc. and was inserted into a pAAV plasmid before the optimized Kozak sequence (GCCACC) and the translation start codon of the ChR2-GFP (FIG. 1A) in an embodiment for expression in human retinal organoids just GFP coding sequence (FIG. 1B), respectively, followed by a woodchuck hepatitis virus posttranscriptional regulatory element (WPRE) and SV40 polyadenylation site.

AAVs were made as described in Grieger, J.C. et al. Production and characterization of a deno-associated viral vectors. Nat. Protoc. 1(2006): 1412-1428.

Genome copy (GC) number titration was performed using real-time PCR (Applied Biosystems, TagMan reagents). Human retinal explants and mouse retina were targeted using AAV serotype BP2 with a titer of 5.15E+13 GC/mL. To retinal organoids serotype PHP.eB with a titer of 4.5E+13 GC/mL was applied.

### Preparation and AAV Infection of Human Retinal Organoids

Retinal organoids were generated from isolated human induced pluripotent stem cells (iPSCs) as described in Zhong et al. Generation of three-dimensional retinal tissue with functional photoreceptors from human iPSCs. Nat. Commun. 5(2014): 4047. In brief, on day zero of differentiation, floating embryoid bodies (EBs) were generated by dissociating iPSC colonies. The EBs were cultured in suspension in mTesR1 medium supplemented with 10 μmol/L ATPase inhibitor Blebbistatin (Sigma, #B0560-5MG).

On days 1 and 2, one third of the medium was exchanged with ‘neural induction medium’ (NIM) containing DMEM/F12 (GIBCO, #31331-028), 1×N2 Supplement (GIBCO, #17502-048), 1% NEAA Solution (Sigma, #M7145) and 2 μg/mL heparin (Sigma, #H3149-50KU).

On day 3, to remove dead cells and debris, EBs were sedimented by gravity, washed with NIM, and cultured in a 3.5 cm untreated Petri dish (Corning, #351008) in NIM. Half of the NIM was exchanged daily. On day 7, EBs from one 3.5 cm dish were plated onto a 6 cm dish (Corning, #430166) coated with Growth Factor-Reduced Matrigel (Corning, #356230) and then maintained with daily NIM changes.

Retinal organoids were infected at week 26 with 10E+11 GC/organoid of a construct according to the invention: AAV-s1000-GFP (serotype PhP.eB) inducing the expression of enhanced GFP under the control of the new synthetic promoter SEQ ID NO:1.

Individual organoids were placed in a single well of an ultra-low attachment U-bottom 96-well plate (Corning, #7007) and maintained at 37° C. in 5% CO2 in 30 μL culturing media including 10E+11 GC of the AAV. After 5 hours, 70 μL of fresh media was added to each well. One day later, 100 μL of fresh media was added to each well. After 24 hours, and every 48 hours thereafter the solution was completely exchanged with fresh media. Infected organoids were cultured for 4 weeks in DMEM (GIBCO, #10569-010) supplemented with 20% Ham's F12 Nutrient Mix (GIBCO, #31765-027), 10% heat-inactivated fetal bovine serum (Millipore, #es-009-b), 1% N2 Supplement (GIBCO, #17502-048), 1% NEAA Solution (Sigma, #M7145), 100 μmol/L taurine (Sigma, #T0625), and 1 μmol/L retinoic acid (Sigma, #R2625).

Organoids were fixed for 4 hours at 4° C. in 4% PFA in PBS. After fixation, samples were washed 3×30 minutes with PBS and cryopreserved in 30% sucrose in PBS overnight at 4° C. Samples were stored at −80° C. until use. Cryosections (20-40 μm) were generated using a cryostat (MICROM International, #HM560) on organoids embedded in O.C.T compound (VWR, #25608-930). Sections were mounted onto glass slides, dried for 4 to 16 hours at room temperature and stored at −80° C. until use. For immunostainings of cryosections, slides were first dried for 30 minutes at room temperature and then rehydrated for 5-10 minutes in PBS. Second, slides were blocked with 10% normal donkey serum (Sigma, #530-100 ML), 1% (wt/vol) bovine serum albumin (BSA; Sigma, #05482-25 G), 0.5% Triton X-100 in PBS (Sigma, #T9284-500 ML) and 0.02% sodium azide (Sigma, #S2002-25 G) in PBS at room temperature for 1 h.

Sections were then incubated in a humidified chamber with primary antibodies (rabbit anti-GFP Ab (Invitrogen; 1:200), mouse monoclonal anti-human cone-arrestin 7G6 (CAR, Zhang, H. et al. Identification and Light-Dependent Translocation of a Cone-Specific Antigen, Cone Arrestin, Recognized by Monoclonal Antibody 7G6. Invest. Ophthalmol. Vis. Sci. 44(2003): 2858-2867) and polyclonal goat anti-ChAT (Millipore: 1:200)). For each slide, primary antibodies were diluted in 100 μL of PBS supplemented with 3% normal donkey serum, 1% BSA, 0.5% Triton X-100 in PBS and 0.02% sodium azide overnight at room temperature. After washing 3×15 minutes in PBS with 0.1% Tween 20 (Sigma, #P9416-100 ML), slides were incubated with secondary antibodies (Thermo Fisher Scientific, donkey secondary antibodies conjugated to Alexa Fluor 488, 568 or 647) diluted 1:200 and Hoechst (1:1000) for two hours at room temperature in the dark. The sections underwent washes of 2×15 minutes in PBS with 0.1% Tween, one wash of 15 minutes in PBS, and were mounted with ProLong Gold (Thermo Fisher Scientific, #P36934). Images were acquired with a spinning disc microscope (Olympus IXplore Spin confocal spinning disc microscope system). Cell-type morphologies were assessed from 1024×1024 pixel images in a z-stack with 0.85 μm z-steps. Images were processed using the Imaris software (v.9.0.2, Bitplane) and ImageJ.

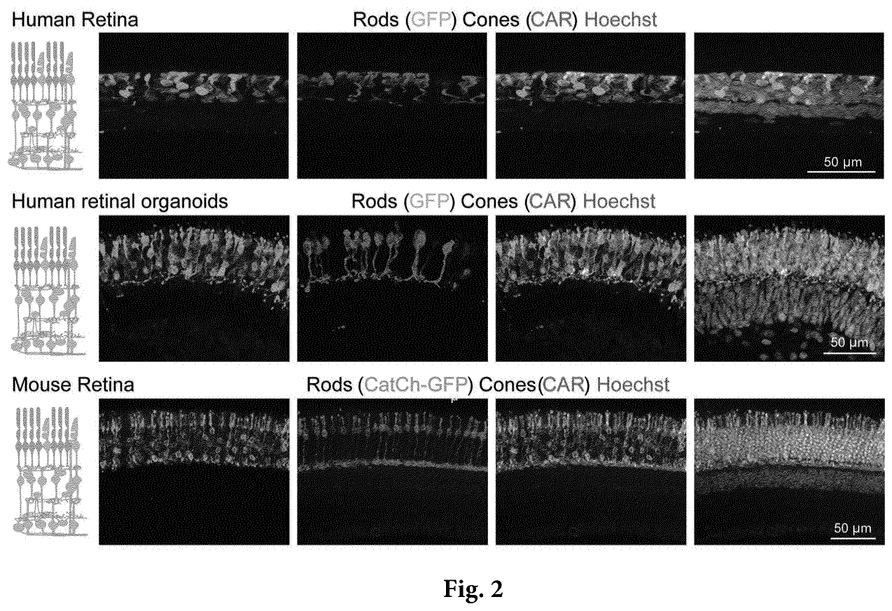

FIG. 2 middle row shows the Spinning disk confocal microscope images of preparations of human organoids which had been infected with AAV-SEQ ID NO:1-Catch-GFP and AAV-SEQ ID NO:1-GFP, respectively. Leftmost column depicts a schematic drawing of the histological structure of the dissected tissue; second column shows the typical green fluorescence conferred by GFP in rod photoreceptors all transfected with CatCh-GFP/GFP; third column shows the result of an immunostaining of the same tissue with cone marker CAR (magenta fluorescence); forth column shows the result of transfection with CatCh-GFP/GFP and cone marker CAR staining: no green fluorescence of CatCh-GFP/GFP can be found in cells showing the cone marker CAR staining; fifth (rightmost) column shows the result of transfection with CatCh-GFP/GFP and cone marker CAR staining right: CatCh-GFP/GFP and cone marker CAR staining and nuclear staining with Hoechst (white).

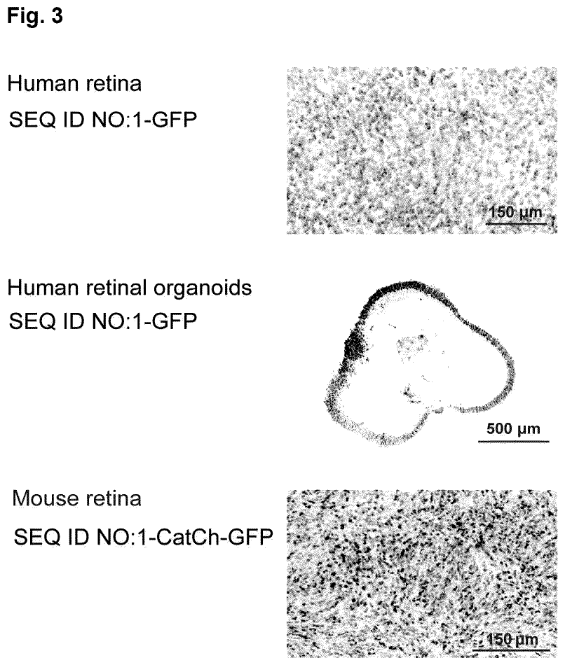

FIG. 3 shows confocal images of CatCh-GFP/GFP-AAV-infected whole human retinal organoid cross-sections, GFP fluorescence is depicted as black staining.

### Viral Transfection and Tissue Preparation Of Human Retina

For human organotypic culture, retina pieces were isolated in cold DMEM/F12 medium, placed ganglion cells side up on 0.4 μm pore polycarbonate membrane (Corning) inserted in 24 mm culture dish, and maintained at 37° C. in 5% CO2 in 1 mL of a culture medium: DMEM/F12 supplemented with 0.1% BSA, 10 μmol/L O-acetyl-L-carnitine hydrochloride, 1 mmol/L fumaric acid, 0.5 mmol/L galactose, 1 mmol/L glucose, 0.5 mmol/L glycine, 10 mmol/L HEPES, 0.05 mmol/L mannose, 13 mmol/L sodium bicarbonate, 3 mmol/L taurine, 0.1 mmol/L putrescine dihydrochloride, 0.35 μmol/L retinol, 0.3 μmol/L retinyl acetate, 0.2 μmol/L (+)-alpha-tocopherol, 0.5 mmol/L ascorbic acid, 0.05 μmol/L sodium selenite, 0.02 μmol/L hydrocortisone, 0.02 μmol/L progesterone, 1 μmol/L insulin, 0.003 μmol/L 3,3′,5-triiodo-L-thyronine, 2,000 U penicillin, and 2 mg streptomycin.

For AAV infection, 20 μL of a construct according to the present invention AAVBP2-s1000 -GFP was applied per retina piece.

AAV-induced transgene expression was examined five weeks after virus administration. After the retinal pieces were fixed for 30 min in 4% PFA in PBS, followed by a washing step in PBS at 4 C, they were treated with 10% normal donkey serum (NDS), 1% BSA, 0.5% Triton X-100 in PBS for 1 h at room temperature. Treatment with monoclonal rabbit anti-GFP Ab (Invitrogen; 1:200), mouse monoclonal anti-human CAR 7G6 (Zhang, H. et al. Identification and Light-Dependent Translocation of a Cone-Specific Antigen, Cone Arrestin, Recognized by Monoclonal Antibody 7G6. Invest. Ophthalmol. Vis. Sci. 44(2003): 2858-2867) and polyclonal goat anti-ChAT (Millipore: 1:200) in 3% NDS, 1% BSA, 0.5% Triton X-100 in PBS was carried out for 5 days at room temperature. Treatment with secondary donkey anti-rabbit Alexa Fluor-488 Ab (Invitrogen; 1:200), anti-goat Alexa Fluor-633 and Hoechst (1:1000), was done for 2 hr. Sections were washed, mounted with ProLong Gold antifade reagent (Thermo Fisher Scientific, #P36934) on glass slides, and imaged using an Olympus IXplore SpinSR confocal microscope (Olympus Corp.). Cell-type morphologies were assessed from 512×512 pixel images in a z-stack with 0.85 μm z-steps. Images were processed using the Imaris software (v.9.0.2, Bitplane) and ImageJ.

FIG. 2 upper row shows the Spinning disk confocal microscope images of human retinae which had been infected with AAV-SEQ ID NO:1-Catch-GFP and AAV-SEQ ID NO:1-GFP, respectively. Leftmost column depicts a schematic drawing of the histological structure of the dissected tissue; second column shows the typical green fluorescence conferred by GFP in rod photoreceptors all transfected with CatCh-GFP/GFP; third column shows the result of an immunostaining of the same tissue with cone marker CAR (magenta fluorescence); forth column shows the result of transfection with CatCh-GFP/GFP and cone marker CAR staining: no green fluorescence of CatCh-GFP/GFP can be found in cells showing the cone marker CAR staining; fifth (rightmost) column shows the result of transfection with CatCh-GFP/GFP and cone marker CAR staining right: CatCh-GFP/GFP and cone marker CAR staining and nuclear staining with Hoechst (white).

FIG. 3 shows confocal images of CatCh-GFP/GFP AAV-infected human retinae (top view), GFP fluorescence is depicted as black staining.

### AAV Administration and Tissue Preparation of Mouse Retina

Ocular injections were performed on 8 weeks old wild type C57BL/6J mice anesthetized with 2.5% isoflurane. A small incision was made with a sharp 30-gauge needle in the sclera near the lens and 2 μL of AAVBP2-s1000-Catch-GFP suspension was injected through this incision into the subretinal/intravitreal space using a blunt 5-μL Hamilton syringe held in a micromanipulator.

3 weeks post AAV injection retinas were dissected from the eyecup and fixed for 30 min in 4% (wt/vol) paraformaldehyde in PBS and washed with PBS for 24 h at 4° C. To improve antibody penetration, retinas were subjected to freeze/thaw cycles after cryoprotection with 30% (wt/vol) sucrose. After washing in PBS, retinal wholemounts were incubated for 2 h in blocking buffer containing 10% (vol/vol) normal donkey serum (Chemicon), 1% (wt/vol) bovine serum albumin (BSA), 0.5% (vol/vol) TritonX-100, and 0.01% sodium azide (Sigma) in PBS. Primary antibody treatment with rat monoclonal anti-GFP (Nacalai, RRID: AB_2313654), rabbit polyclonal anti-mouse CAR (Millipore, RRID: AB_1163387), goat polyclonal anti-ChAT (Millipore, RRID: AB_2079751) diluted 1:200 was performed for 5 days at room temperature in buffer containing 3% (vol/vol) NDS, 1% (wt/vol) BSA, 0.01% (wt/vol) sodium azide, and 0.5% TritonX-100 in PBS. Secondary antibody incubation with Alexa Fluor 568 donkey anti-rabbit IgG (H+L, RRID:AB_2534017), Alexa Fluor 488 donkey anti-rat IgG (H+L, RRID: AB_141709), Alexa Fluor 568 donkey anti-goat IgG (H+L, RRID: AB_142581) was performed for 2 h at room temperature in buffer supplemented with Hoechst 33342 (10 μg/mL). After washing in PBS, retinas were embedded in Prolong Gold antifade (Thermo Fisher Scientific, #P36934). Images were acquired with a spinning disc microscope (Olympus IXplore Spin confocal spinning disc microscope system). Cell-type morphologies were assessed from 1024×1024 pixel images in a z-stack with 0.85 μm z-steps. Images were processed using the Imaris software (v.9.0.2, Bitplane) and ImageJ.

FIG. 2 lower row shows the Spinning disk confocal microscope images of cross-sections of the whole mount mouse retinae which had been infected with AAV-SEQ ID NO:1-Catch-GFP and AAV-SEQ ID NO:1-GFP, respectively. Leftmost column depicts a schematic drawing of the histological structure of the dissected tissue; second column shows the typical green fluorescence conferred by GFP in rod photoreceptors all transfected with CatCh-GFP/GFP; third column shows the result of an immunostaining of the same tissue with cone marker CAR (magenta fluorescence); forth column shows the result of transfection with CatCh-GFP/GFP and cone marker CAR staining: no green fluorescence of CatCh-GFP/GFP can be found in cells showing the cone marker CAR staining; fifth (rightmost) column shows the result of transfection with CatCh-GFP/GFP and cone marker CAR staining right: CatCh-GFP/GFP and cone marker CAR staining and nuclear staining with Hoechst (white).

Furthermore FIG. 3 shows confocal image of AAV-infected mouse retina (top view) with CatCh-GFP/GFP (black staining).

### Analysis

Expression density was defined as the percentage density of labeled rod photoreceptors relative to published mean density of rod photoreceptors (cells/mm²) in human (Jonas, J. B. et al. Count and density of human retinal photoreceptors. Graefes Arch. Clin. Exp. Ophthalmol. 230(1992):505-510) and in mouse retina (Jeon, C. J. et al. The Major Cell Populations of the Mouse Retina. J. Neurosci. 18(1998):8936-8946).

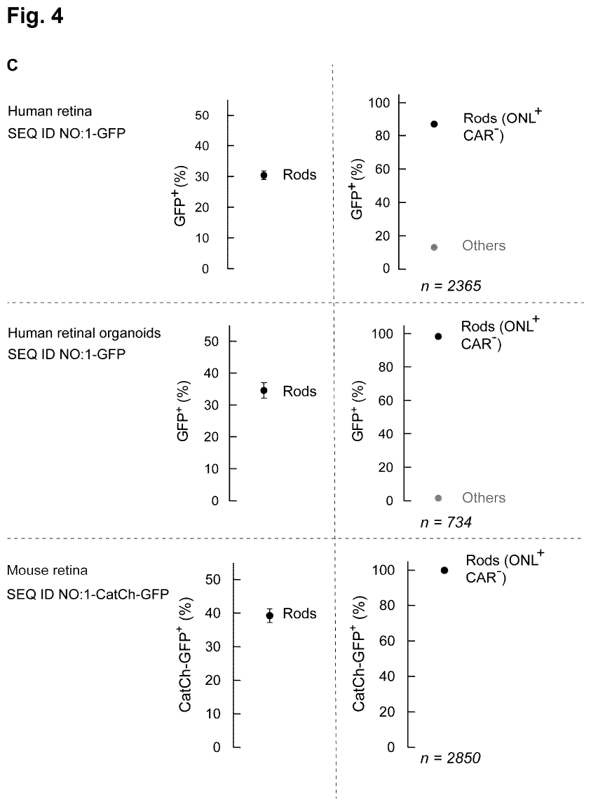

FIG. 4 shows quantification plots for expression in human retina (top row), human retinal organoids (middle row) and mouse retina (lower row): left col.: quantification of CatCh-GFP/GFP+ cell density as a percentage of rod photoreceptor density; values are the mean±s.e.m. from n=12 confocal images; right col:: quantification of AAV-targeting specificity shown as a percentage of the major (black) and minor (gray) cell types or classes among cells expressing CatCh-GFP/GFP.

The synthetic promoter SEQ ID NO:1 drove expression in 30.4% of total human rod photoreceptors and 41.2% of total murine rod photoreceptors. In retinal organoids we quantified the total number of rod photoreceptors/mm² in cryosections stained with rod marker NRL and calculated that SEQ ID NO:1 drove expression in 34.6% of NRL+ cells in retinal organoids (FIG. 4, left column).

Expression specificity was quantified by determining the percentage of rod photoreceptors in the overall GFP+ cell population highlighted by the AAV. Expression in rod photoreceptor was identified by the position of the cell bodies in the retinal outer nuclear layer and the absence of cone arrestin (CAR) marker expression (Zhu, Mol Vis 8(2002):462-471. http://www.molvis.org/molvis/v8/a56/).

In human retina and human retinal organoids GFP expression was observed in 86.9% and 100% of rod photoreceptors, respectively. In mouse retina 98.4% of the observed Catch-GFP+ cells were rod photoreceptors (FIG. 4, right column).

The promoter sequence SEQ ID NO:1 embedded into an AAV drives specific and efficient gene expression in rod photoreceptors of mice, organoids and humans. This allows basic and preclinical research specific manipulation of rod photoreceptors in economic model systems as in vivo mouse retina and in vitro human retinal organoids to discover retinal disease mechanisms and develop new gene therapy approaches. The ability to use of the same viral vector on human retina allows direct translation of these results and will therefore accelerate the application of novel gene therapies in the clinic.

1. An isolated nucleic acid molecule, comprising or consisting of:
a first nucleic acid sequence having promoter activity for the specific expression of an exogenous gene in rod photoreceptor cells and
at least one further nucleic acid sequence encoding the exogenous gene, operably linked to said promoter,
wherein said first nucleic acid sequence comprises or consists of SEQ ID NO:1 or of a functionally equivalent sequence variant thereof, having at least 80% sequence identity to SEQ ID NO:1, and is operably linked to the further nucleic acid sequence.

2. The isolated nucleic acid of claim 1, wherein the first nucleic acid and the further nucleic acid are heterologous.

3. The nucleic acid molecule of claim 1 or 2, wherein the first nucleic acid sequence has a sequence length of 800 to 1000 bp.

4. The nucleic acid molecule of any one of the preceding claims, wherein the at least one further nucleic acid sequence encodes for one or more of: therapeutically effective proteins, peptides or nucleic acids, or a reporter molecule.

5. An expression cassette for the specific expression of an exogenous gene in rod photoreceptor cells, comprising or consisting of the nucleic acid molecule of any one of the preceding claims.

6. A viral vector for the specific expression of an exogenous gene in rod photoreceptor cells, comprising the expression cassette of claim 5.

7. The viral vector of claim 6, which is an adeno-associated viral (AAV) vector or an AVV-derived hybrid vector.

8. A viral particle, comprising an AVV-derived capsid and the AAV vector of claim 7.

9. A host cell transformed with the expression cassette of claim 5.

10. A pharmaceutical composition comprising:
an expression cassette of claim 5, a vector of any one of claims 6 to 7, a viral particle of claim 8, or a transformed cell of claim 8, and
a pharmaceutically acceptable carrier or excipient.

11. An expression cassette of claim 5, a vector of any one of claims 6 to 7, a viral particle of claim 8, a transformed cell of claim 9, or a pharmaceutical composition of claim 10, for use in the treatment or prevention of an ocular disease in a human or animal.

12. The expression cassette, vector, cell, viral particle, or pharmaceutical composition for use of claim 11, wherein the ocular disease is a disease associated with rod photoreceptor cell degeneration and is selected from: age-related macular degeneration, Leber's hereditary optic neuropathy, rod-cone dystrophy, rod dysfunction, Leber congenital amaurosis, Stargardt's disease, diabetic retinopathy, Best's disease, Retinitis Pigmentosa, Choroideremia or a tapetoretinal degeneration.

13. Use of a nucleic acid molecule having promoter activity and as defined in claim 1 or 2 or 3 for the specific expression of a therapeutically effective protein, peptide or nucleic acid, or a reporter molecule in rod photoreceptor cells.

14. Method for the specific transformation of a rod photoreceptor cell with a therapeutically effective protein, peptide or nucleic acid, or a reporter molecule, comprising the step of incubating the rod photoreceptor cell with or introducing in the rod photoreceptor cell:
an expression cassette of claim 5,
a vector of any one of claims 6 to 7,
a viral particle of claim 8,
a transformed cell of claim 9, or
a pharmaceutical composition of claim 10.

15. A kit for expressing a gene in rod photoreceptors comprising an isolated nucleic acid molecule according to claim 1.
